(19) United States
(12) Patent Application Publication
(10) Pub. No.: US 2009/0311315 A1
(43) Pub. Date: Dec. 17, 2009

(54) Multimicroparticulate Oral Pharmaceutical Form with Modified Release of Angiotensin II Receptor Antagonists

(75) Inventors: Catherine Castan, Orlienas (FR); Florence Guimberteau, Montussan (FR); Rémi Meyrueix, Lyon (FR); Gérard Soula, Meyzieu (FR)

Correspondence Address: PATTON BOGGS LLP, 8484 WESTPARK DRIVE, SUITE 900, MCLEAN, VA, 22102, US

(73) Assignee: FLAMEL TECHNOLOGIES, S.A., VENISSIEUX CEDEX (FR)

(21) Appl. No.: 11/884,549

(22) PCT Filed: Feb. 21, 2006
(86) PCT No.: PCT/EP2006/060159

§ 371 Date: Feb. 27, 2009

(30) Foreign Application Priority Data
Feb. 21, 2005 (FR) 0550478

(51) Int. Cl. A61K 9/52; A61K 9/14; A61K 9/22; A61K 31/4178; A61K 31/4184; A61K 9/26

(52) U.S. Cl. 424/456; 424/489; 424/490; 514/397; 514/381; 424/469; 424/470

(57) ABSTRACT

The invention relates to oral pharmaceutical forms with modified release of ARB, and to related treatments and delivery methods.

The invention concerns a form with modified release of ARB which prolongs the bioabsorption time and enables the pharmaceutical form to be administered only once daily.

Therefore, the invention is an oral pharmaceutical form with modified ARB release comprising a plurality of ARB microunits (mean diameter: 50-1000 μm) leading, after being taken, to a plasma profile wherein C18 h*≦C18 h, with C18 h=plasma ARB concentration, 18 h after being taken, C18 h*=plasma ARB concentration corresponding to C18 h and obtained under the same conditions as C18 h, with a reference immediate-release oral pharmaceutical form*, containing the same dose of ARB, Cmax=maximum plasma ARB concentration after being taken, Cmax*=maximum plasma ARB concentration corresponding to Cmax and obtained under the same conditions as Cmax, with a reference immediate-release oral pharmaceutical form*, containing the same dose of ARB.

### FIELD OF THE INVENTION

The field of the present invention is that of oral pharmaceutical forms with modified release of angiotensin II receptor antagonists [or ARB (Angiotensin Receptor Blocker)], and also related treatments and administration methods. By convention, the acronym “ARB” used in the singular in the present disclosure will denote without distinction one or more ARB per se and/or at least one of the pharmaceutically acceptable salts or esters thereof and/or at least one of the active metabolites thereof, with the exclusion of losartan. The expression “losartan” denotes losartan per se and/or at least one of the pharmaceutically acceptable salts or esters thereof.

### GENERALITIES REGARDING ARB

ARB are active by oral administration and are involved in the regulation of hypertension by acting on the renin-angiotensin system.

As nonlimiting examples of ARB, mention may be made of:

irbesartan, olmesartan, eprosartan, candesartan, candesartan cilexetil, valsartan, telmisartan, zolasartin and tasosartan.

ARB can be combined with a diuretic (hydrochlorothiazide) in order to increase their efficacy.

ARB are in particular used in the treatment of the following pathologies:

essential arterial hypertension,

treatment of renal insufficiency in type 2 diabetic patients with proteinuria,

reduction of cardiovascular morbidity and mortality in hypertensive patients with left ventricular hypertrophy (most commonly in combination with a thiazide diuretic),

congestive heart failure

polyglobulia in patients having received a kidney transplant.

### PROBLEMATIC

The problems posed by the oral administration of ARB are in particular the following.

### Problem 1:

The arterial pressure of patients after oral administration of ARB is tightly linked to the plasma ARB concentration. Now, from 12 to 18 hours onwards after intake, the plasma ARB concentration is low. This is due to the low value of the elimination half-life. Thus, for example, the elimination half-lives of eprosartan cilexitil, cardesartan and valsartan are, respectively, 5-9 h, 4-9 h and 4-6 h.

It would therefore be recommended to administer the ARB-based oral medicaments several times a day. However, it is commonly accepted that a posology involving several intakes per day is not recommended in terms of adherence to the treatment and therefore effectiveness of the treatment.

It is therefore desirable to have a modified-release form of ARB which prolongs the bioabsorption time and which makes it possible to administer the medicament only once daily.

### Problem 2:

The guarantee of therapeutic safety represents high stakes, in particular for antihypertensives such as ARB. In fact, it is essential to be able to have an oral medicament designed in such a way that, once ingested, the active ingredient that it contains is released in the gastrointestinal tract and bioabsorbed in its window of absorption. Failing this, the dose of active ingredient is evacuated with the intestinal transit without being correctly absorbed. It does not therefore produce the expected therapeutic effect. In the case of ARBs, the arterial pressure of the patient having swallowed the tablet is not reduced, which considerably increases the risks of infarction and thus endangers the patient's life.

The literature describes modified-release ARB forms: gastroretentive tablets with sustained release. The tablet swells in the stomach to a size which, when the pylorus is closed (i.e. in the fed state), prevents gastric emptying thereof. The active ingredient is thus released gradually, upstream of its window of absorption located in the upper parts of the small intestine. It can therefore be absorbed correctly.

However, for a not insignificant fraction of patients, the closing of the pylorus can be erratic. In addition, if the patient does not scrupulously observe the posological recommendations and ingests the tablet before a meal or at the beginning of a meal, before the pylorus is closed, it is possible for the swallowed tablet not to stay in the stomach and to be rapidly evacuated without having released the active ingredient upstream of its window of bioabsorption.

These sustained-release gastroretentive forms are not therefore safe, since the active ingredient is not necessarily bioabsorbed, whether the patient is in the fed state or in the unfed state.

It is therefore essential for a modified-release oral form to be able to make sure that, once the oral medicament has been ingested, the active ingredient is bioabsorbed, whether the patient is in the fed state or in the unfed state.

Consequently, the ideal situation would be to have an oral pharmaceutical form of ARB:

which makes sure that, once the oral pharmaceutical form has been ingested, the ARB that it contains is released in the gastrointestinal tract and bioabsorbed in its window of gastrointestinal absorption, whether the patient is in the fed state or in the unfed state, i.e. which allows good reproducibility of the plasma concentration, by limiting—or even by eliminating—the harmful effects of the interindividual variability of gastric emptying,

which can be administered once daily,

which is more effective than the immediate-release “once daily” forms.

### PRIOR ART

### Monolithic Forms

Patent application WO-A-03/035039 describes a controlled-release galenic form of losartan. The basis of the invention according to WO-A-03/035039, is to propose a gastroretentive galenic form which can be administered once daily.

This galenic form consists of a tablet of “bioadhesive” gastroretentive type which can swell so as to reach a size sufficient to be retained in the fed stomach. This controlled-release galenic form of losartan is large in size, monolithic and multilayered (2, possibly 3), based on polymers of hydroxypropylmethylcellulose (HPMC) or polyethylene oxide (PEO) type, capable of swelling in the gastric medium. The losartan can be in the form of a single core or of a plurality of particles (page 8, lines 29, 30) included in a compressed HPMC/PEO polymer matrix. A nonactive layer formed by HPMC/PEO swelling polymers can be applied to this matrix (or active layer), also by compression.

The pharmacokinetic parameters, measured in dogs, of the two gastroretentive forms GR1 and GR2 exemplified are given in table 1 below:

TABLE 1

| | | Controlled | Controlled |
|---|---|---|---|
| | | release by | release by |
| | Immediate | gastroretention | gastroretention |
| Parameters | release | GR1 | GR2 |
| AUC (ng/ml · h) | 486 ± 133 | 590 ± 202 | 461 ± 176 |
| Cmax (ng/ml) | 224 ± 58.5 | 105 ± 31.7 | 72 ± 24.1 |
| Tmax (h) | 0.88 ± 0.25 | 2.5 ± 0.58 | 5.25 ± 0.5 |
| GR time (h) | / | 7.6 ± 2.5 | 6.8 ± 0.5 |

Cmax = maximum plasma concentration after oral administration
Tmax = time elapsed after oral administration and corresponding to Cmax
GR = gastroretention

First of all, the sustained-release gastroretentive tablet according to WO-A-03/035039 is deficient in terms of guaranteeing therapeutic safety (problem No. 2 above). In fact, it is not certain that, once the oral medicament has been ingested, the losartan that it contains is released and bioabsorbed in its window of absorption. The expected therapeutic action may not occur, resulting in the patient's life being endangered. In particular, depending on whether the patient is in the fed state or in the unfed state, the gastroretentive tablet according to WO-A-03/035039 can escape from the stomach prematurely and be rapidly evacuated without having released the losartan, which is not therefore bioabsorbed and which thus does not produce the expected effect on limiting the arterial pressure. This goes back to the major drawback of interindividual variability of gastric emptying, of gastroretentive monolithic forms.

In addition, this gastroretentive tablet can be considerable in size. It cannot be fragmented in order to facilitate its ingestion without harming the characteristics of modified release of the losartan. It is therefore unsuitable for patients who have difficulty in swallowing and even less so for children or infants who are no better at swallowing and who, in addition, impose an adjustment of the administered dose according to their weight.

Furthermore, it is difficult to mix losartan with one or more active ingredients in the same tablet, and it is even more difficult to independently adjust the release times of the various active ingredients.

Moreover, with a losartan tablet, the risk of tissue deterioration due to a local overconcentration of losartan, which is well known to be aggressive, cannot be discarded.

Thus, inescapably, unacceptable therapeutic risks therefore persist for the gastroretentive tablets according to WO-A-03/035039.

### Multimicroparticulate Forms

PCT patent application WO-A-03/020243 discloses tablets or gel capsules comprising an anticholesterol agent, an angiotensin II receptor antagonist (ARB), aspirin and, optionally, vitamin B6 or B12 or a folate. This formulation is intended for the prevention of the cardiovascular risk in patients with a high cardiovascular risk. The ARB may be losartan. The ARB, like the other active agents, can be in the form of beads, particles or granules (e.g. 14-26 mesh: 700-1410 μm) coated (enteric coating: EUDRAGIT® L 30D-55 and diethyl phthalate) so as to allow the delayed or sustained release of the ARB.

This prior technical proposal does not make it possible to solve the abovementioned technical problems.

### OBJECTIVES

Bolstered by these observations, the applicant assigned itself the following objectives.

The essential objective of the invention is to remedy the insufficiencies and drawbacks of the prior art by proposing a modified-release form of ARB which prolongs the bioabsorption time and which makes it possible to administer the medicament only once daily.

Another essential objective of the invention is to provide an oral pharmaceutical form of ARB designed in such a way that, once the oral pharmaceutical form has been ingested, the ARB that it contains is released in the gastrointestinal tract and bioabsorbed in its window of absorption.

Another essential objective of the invention is to provide an oral pharmaceutical form of ARB, which reduces the repercussions of the interindividual variability of the in vivo absorption of ARBs, which is a direct consequence of the sensitivity of certain oral galenic forms with respect to the interindividual variability of gastric emptying.

Another essential objective of the invention is to provide an oral pharmaceutical form of ARB which can be administered once daily and is at least as effective as the immediate-release once-daily forms currently in use.

Another essential objective of the invention is to provide an oral pharmaceutical form of ARB which has an in vitro dissolution profile independent of the dose of ARB.

Another essential objective of the invention is to provide an oral pharmaceutical form of ARB which has the same composition by weight irrespective of the intended therapeutic dose of ARB.

Another essential objective of the invention is to provide an oral pharmaceutical form of ARB which can be administered once daily and is suitable for patients who have difficulties in swallowing, in particular for children and infants who not only cannot swallow, but who, in addition, require the administered dose to be adjusted according to their weight.

Another essential objective of the invention is to provide an oral pharmaceutical form of ARB which can be administered once daily and which offers the possibility of mixing the ARB with one or more active ingredients in the same oral form, with the possibility of independently readily adjusting the release times of the various active ingredients.

Another essential objective of the invention is to provide an oral pharmaceutical form of ARB which can be administered once daily and which limits the risk of tissue deterioration due to local overconcentration of ARB.

Another essential objective of the invention is to provide an oral pharmaceutical form of ARB which can exist in various galenic presentation forms, including in particular: tablet, sachet, oral suspension, gel capsule or the like.

### SUCCINCT DESCRIPTION OF THE INVENTION

In this context, it is first of all to the applicant's credit

to have identified the abovementioned problems 1 and 2 and their causes,

to have developed a modified-release multimicroparticulate form which has suitable pharmacokinetic characteristics.

In order to achieve these objectives, among others, it is to the applicant's credit to have developed a multimicroparticulate oral pharmaceutical form of ARB which results in gradual release of the ARB in a region of the gastrointestinal tract where the ARB is bioabsorbable.

Thus, the present invention proposes a novel oral pharmaceutical form with modified release of ARB, characterized: in that it comprises a plurality of microunits containing ARB,

in that the average diameter (Dm in μm) of the microunits is between 50 and 1000, preferably 100 and 600, and even more preferably between 150 and 500,

and in that it makes it possible to obtain, after one intake, a plasma profile defined as follows:

| | |
|---|---|
| | C18 h* ≦ C18 h |
| preferably | 1.5 × C18 h* ≦ C18 h ≦ Cmax*/1.5 |
| and even more | 2.0 × C18 h* ≦ C18 h ≦ Cmax*/1.5 |
| preferably | |

with
C18 h representing the plasma concentration of ARB, 18 h after the intake,
C18 h* representing the plasma concentration of ARB obtained under the same conditions as C18 h, with a reference immediate-release oral pharmaceutical form, containing the same dose of ARB,
Cmax representing the maximum plasma concentration of ARB after the intake,
Cmax* representing the maximum plasma concentration of ARB obtained under the same conditions as Cmax, with a reference immediate-release oral pharmaceutical form, containing the same dose of ARB.

According to one variant, this novel oral pharmaceutical form with modified release of ARB is characterized:

in that it comprises a plurality of microunits containing ARB,

in that the average diameter (Dm in μm) of the microunits is between 50 and 1000, preferably 100 and 600, and even more preferably between 150 and 500,

and in that it makes it possible to obtain, after one intake, a plasma profile defined as follows:

| | |
|---|---|
| | C18 h* ≦ C18 h |
| preferably | 1.5 × C18 h* ≦ C18 h ≦ Cmax*/1.5 |
| and even more | 2.0 × C18 h* ≦ C18 h ≦ Cmax*/1.5 |
| preferably | |
| and | 1.1.Tmax* ≦ Tmax |
| preferably | 1.2.Tmax* ≦ Tmax |
| and more preferably | 1.5.Tmax* ≦ Tmax |
| even more preferably | 1.7.Tmax* ≦ Tmax ≦ 6.Tmax |

with
C18 h representing the plasma concentration of ARB, 18 h after the intake,
C18 h* representing the plasma concentration of ARB obtained under the same conditions as C18 h, with a reference immediate-release oral pharmaceutical form, containing the same dose of ARB,
Cmax representing the maximum plasma concentration of ARB after the intake,
Tmax representing the time which has elapsed after the intake and which corresponds to Cmax,
Cmax* representing the maximum plasma concentration of ARB obtained under the same conditions as Cmax, with a reference immediate-release oral pharmaceutical form, containing the same dose of ARB,
Tmax* representing the time which has elapsed after the intake and which corresponds to Cmax*.

This oral pharmaceutical form with modified release of ARB is designed in such a way that the microunits, once ingested, are dispersed and individualized when they reach the stomach, thereby guaranteeing regular and gradual gastric emptying of the microunits, in the fed state as in the unfed state, and therefore, ultimately, release of the ARB in its gastrointestinal window of bioabsorption.

For the purpose of the invention, the expression “dispersed and individualized” means that the ARB-based microunits are not trapped in a matrix when they reach the stomach just after they have been ingested. The microunits are disseminated in the stomach immediately after they have entered the latter (for example, in less than two minutes).

### DETAILED DESCRIPTION

The term “modified release” denotes, in the present disclosure, a release of ARB by a pharmaceutical formulation, this release being carried out at a rate less than that of a reference “immediate-release” formulation IRF*, such as a conventional tablet or gel capsule to be swallowed. Such a modified-release formulation can, for example, comprise an immediate-release phase and a slow-release phase. Modified-release formulations are well known in this field; see, for example, Remington: The science and practice of pharmacy, 19th edition, Mack Publishing Co. Pennsylvania, USA.

The term “immediate release” denotes, in the present disclosure, the release, by an IRF, of the majority of the amount of ARB in a relatively brief period of time, for example 80% in one hour, preferably in thirty minutes, after oral ingestion. Examples of such IRFs comprise conventional tablets to be swallowed, dispersible tablets, chewable tablets, sachets of unit doses, and gel capsules.

The comparison of the parameters C18h and C18h*, Cmax and Cmax*, and also Tmax and Tmax* is carried out in a statistically significant manner, under the same conditions and at the same dose of ARB.

In accordance with the invention, the microunits denote:

microparticles coated with at least one coating which allows the modified release of ARB, and

microgranules with immediate release of ARB.

The advantages of the invention are in particular the following:

This oral pharmaceutical form of ARB, which can be administered once daily, is such that, once ingested, the ARB that it contains is released in the gastrointestinal tract and bioabsorbed in its window of absorption, even if the latter is narrow.

This oral pharmaceutical form of ARB, which can be administered once daily, guarantees that, once the oral pharmaceutical form has been ingested, the ARB that it contains will not pass in front of its window of bioabsorption without being released.

This oral pharmaceutical form of ARB, which can be administered once daily, guarantees that, once the oral pharmaceutical form has been ingested, the ARB that it contains will be released independently of the open or closed state of the pylorus.

This oral pharmaceutical form of ARB, which can be administered once daily, is barely or not at all subject to the phenomenon of interindividual variability of gastric emptying and, ultimately, of the in vivo absorption of the ARB.

This oral pharmaceutical form of ARB, which can be administered once daily, is at least as effective as the immediate-release, once-daily forms currently in use.

This oral pharmaceutical form of ARB, which can be administered once daily and which comprises microunits with modified release of ARB, draws some of its advantages from the small size (50-1000 um) of these microunits and the large number thereof (e.g. several thousand per dose), which allows a gradual and well-controlled gastric emptying, independently of whether the patients have eaten.

This oral pharmaceutical form of ARB, which can be administered once daily, makes it possible to increase the Tmax of the ARB and also the period during which the plasma concentration of ARB is greater than the floor plasma concentration of ARB, below which the ARB is therapeutically ineffective.

This oral pharmaceutical form of ARB has an in vitro dissolution profile independent of the dose of ARB.

This oral pharmaceutical form of ARB can have the same composition by weight irrespective of the doses of ARB.

This oral pharmaceutical form of ARB, which can be administered once daily, is suitable for patients who have difficulties in swallowing, in particular for children or infants who not only cannot swallow solids but who, in addition, require the administered dose to be adjusted according to their weight.

This oral pharmaceutical form of ARB, which can be administered once daily, offers the possibility of mixing the ARB with one or more other active ingredients in the same oral form, it being possible for the respective release times of these various active ingredients to be readily adjusted, independently of one another.

This oral pharmaceutical form of ARB can exist in various galenic presentation forms, including in particular: tablet, sachet, oral suspension, gel capsule, or the like.

The oral galenic form according to the invention consists of a large number (for example, of the order of a thousand to several thousand) of microunits (microparticles or microgranules of ARB), this multiplicity ensuring statistically a good reproducibility of the kinetics of transit of the ARB throughout the gastrointestinal tract, and, subsequently, good control of the bioavailability and better effectiveness.

The use of a mixture of microparticles with different modified-release profiles makes it possible to produce release profiles which have several release waves or which provide, due to appropriate regulating of the various fractions, a constant ARB plasma concentration level.

The sensitivity to variability in gastric emptying is reduced, since the emptying, which here takes place over a large number of particles, is statistically more reproducible.

Contact between the tissues and a high dose of ARB (“dose dumping”) is avoided. Each microunit in fact contains only a very small dose of ARB. The risk of tissue deterioration due to a local overconcentration of aggressive ARB is thus avoided.

This pharmaceutical form does not induce any degradation of the starting ARB and preserves the polymorphism of this starting ARB.

The size of the microunits, which is between 50 and 1000 um, and also the characteristics of their possible coating, allows said microunits to increase their transit time in the upper parts of the gastrointestinal tract, thereby ensuring an increase in the time during which the ARB passes in front of its window of absorption and thus maximizing the bioavailability of the ARB.

Preferably, at least some of the microunits are microparticles individually consisting of a core which comprises the ARB and which is coated with at least one coating which allows the modified release of the ARB.

It can also be very advantageous for at least some of the microunits of the pharmaceutical form according to the invention to consist of microgranules with immediate release of the ARB.

Preferably, the oral pharmaceutical form according to the invention is characterized in that the variability CV (as %) of the area under the curve (AUC) of the plasma concentration of ARB, as a function of time (T) after the intake, is less than or equal to 200%, preferably to 150%, and even more preferably to 120%, of the corresponding variability CV* (as %) of the area under the curve (AUC*) giving the evolution of the plasma concentration of ARB, as a function of time (T) after the intake, under the same conditions, of a reference immediate-release oral pharmaceutical form containing the same dose of ARB, i.e.: CV≦1.5×CV*, preferably CV≦1.2×CV*.

The pharmacokinetic parameters CV and AUC are well known to those skilled in the art.

The comparison of the modified-release form of ARB according to the invention and of the IRF*, in particular of the parameters CV and CV*, and AUC and AUC*, is carried out in a statistically significant manner, under the same conditions and at the same dose of ARB.

All the in vitro dissolution profiles to which reference is made in the present disclosure are carried out according to the indications of the European Pharmacopeia, 4th edition, entitled: “Dissolution test for solid oral forms”: type II dissolutest carried out under SINK conditions, maintained at 37° C. and stirred at 100 rpm.

In accordance with a first embodiment of the invention, the oral pharmaceutical form has an in vitro dissolution profile such that:

70% of the ARB is released between 1 and 24 h, preferably between 2 and 12 h, and even more preferably between 2 and 8 h, after the administration.

The composition of the coating of the microparticles according to the first embodiment corresponds, advantageously, to one of the following two families A and B:

### Family A

1A—at least one film-forming polymer (P1) which is insoluble in the fluids of the tract, present in a proportion of 50 to 90, preferably 50 to 80 by weight on a dry basis, relative to the total mass of the coating composition, and consisting of at least one water-insoluble derivative of cellulose;

2A—at least one nitrogenous polymer (P2) present in a proportion of 2 to 25, preferably 5 to 15% by weight on a dry basis, relative to the total mass of the coating composition, and consisting of at least one polyacrylamide and/or one poly-N-vinylamide and/or one poly-N-vinyllactam;

3A—at least one plasticizer present in a proportion of 2 to 20, preferably 4 to 15% by weight on a dry basis, relative to the total mass of the coating composition, and consisting of at least one of the following compounds: glyceryl esters, phthalates, citrates, sebacates, cetyl alcohol esters, castor oil;

4A—at least one surfactant and/or lubricant, present in a proportion of 2 to 20, preferably 4 to 15% by weight on a dry basis, relative to the total mass of the coating composition, and chosen from anionic surfactants and/or from nonionic surfactants and/or from lubricants; it being possible for said surfactant and/or lubricant to comprise just one or a mixture of the abovementioned products;

### Family B:

1B—at least one film-forming polymer which is insoluble in the fluids of the gastrointestinal tract,

2B—at least one water-soluble polymer,

3B—at least one plasticizer,

4B—and, optionally, at least one surfactant/lubricant, preferably consisting of at least one anionic surfactant and/or at least one nonionic surfactant.

According to a preferred mode of the invention, the families A and B from which the constituents of the coating composition are chosen are as follows:

### Family A

1A—ethylcellulose and/or cellulose acetate;

2A—polyacrylamide and/or polyvinylpyrrolidone;

3A—castor oil;

4A—an alkali metal or alkaline earth metal salt of fatty acids, stearic acid and/or oleic acid being preferred, a polyoxyethylenated sorbitan ester, polyoxyethylenated castor oil derivatives, a stearate, preferably calcium stearate, magnesium stearate, aluminum stearate or zinc stearate, a stearyl fumarate, preferably sodium stearyl fumarate, glyceryl behenate; taken by themselves or as a mixture with one another;

### Family B:

### 1B:

water-insoluble derivatives of cellulose, ethyl-cellulose and/or cellulose acetate being particularly preferred,

acrylic derivatives,

polyvinyl acetates,

and mixtures thereof.

### 2B:

water-soluble derivatives of cellulose,

polyacrylamides,

poly-N-vinylamides,

poly-N-vinyllactams,

polyvinyl alcohols (PVAs),

polyoxyethylenes (POEs),

polyvinylpyrrolidones (PVPs) (the latter being preferred),

and mixtures thereof;

### 3B:

glycerol and its esters, preferably from the following subgroup: acetylated glycerides, glyceryl monostearate, glyceryl triacetate, glyceryl tributyrate,

phthalates, preferably from the following subgroup: dibutyl phthalate, diethyl phthalate, dimethyl phthalate, dioctyl phthalate,

citrates, preferably from the following subgroup: acetyl tributyl citrate, acetyl triethyl citrate, tributyl citrate, triethyl citrate,

sebacates, preferably from the following subgroup: diethyl sebacate, dibutyl sebacate,

adipates,

azelates,

benzoates,

plant oils,

fumarates, preferably diethyl fumarate,

malates, preferably diethyl malate,

oxalates, preferably diethyl oxalate,

succinates, preferably dibutyl succinate,

butyrates,

cetyl alcohol esters,

salicylic acid,

malonates, preferably diethyl malonate,

castor oil (the latter being particularly preferred), and mixtures thereof;

### 4B:

alkali metal or alkaline earth metal salts of fatty acids, stearic acid and/or oleic acid being preferred, polyoxyethylenated oils, preferably polyoxyethylenated hydrogenated castor oil,

polyoxyethylene-polyoxypropylene copolymers, polyoxyethylenated sorbitan esters, polyoxyethylenated castor oil derivatives,

stearates, preferably calcium stearate, magnesium stearate, aluminum stearate or zinc stearate,

stearyl fumarates, preferably sodium stearyl fumarate, glyceryl behenate,

and mixtures thereof.

Preferably, the film coating consists of a single layer, the mass of which represents from 1 to 50% by weight, preferably from 5 to 40% by weight, of the total mass of the microparticles.

Other details and examples of compositions and of methods for obtaining the microparticles according to the first embodiment of the invention are given in WO-A-03/084518, the content of which is integrated into the present disclosure by way of reference.

For further data from the qualitative and quantitative point of view, as regards the coating composition of family A, reference will be made to European patent EP-B-0 709 087, the content of which is integrated into the present disclosure by way of reference.

In accordance with a second embodiment of the invention, the oral pharmaceutical form is such that:

the release of the ARB is controlled by two distinct triggering mechanisms, one being based on a variation in pH and the other allowing the release of the ARB after a predetermined residence time in the stomach;

at a constant pH 1.4, the dissolution profile comprises a lag phase with a duration of less than or equal to 7 hours, preferably less than or equal to 5 hours, and even more preferably of between 1 and 5 hours,

and the passage from pH 1.4 to pH 7.0 results in a release phase which begins with no lag time.

In accordance with the second embodiment of the invention, the pharmaceutical form has an in vitro dissolution profile which can be as indicated hereinafter:

less than 20% of the ARB is released after 2 hours at pH=1.4;

at least 50% of the ARB is released after 16 hours at pH=1.4.

Advantageously, the microparticles with modified release of ARB, according to the second embodiment of the invention, have the following specificities:

the coating which allows the modified release of the ARB comprises a composite material
comprising:
   at least one hydrophilic polymer I bearing groups which are ionized at neutral pH,
   at least one hydrophobic compound II;
representing a mass fraction (% by weight relative to the total mass of the microparticles)≦40; and

their average diameter is less than 2000 μm, and preferably between 50 and 800 um, and even more preferably between 100 and 600 μm.

According to another advantageous characteristic, the composite material I-II of the coating which allows the modified release of ARB is such that:

the weight ratio II/I is between 0.2 and 1.5, preferably between 0.5 and 1.0,

and the hydrophobic compound II is selected from products which are crystalline in the solid state and have a melting temperature T_{mII}≧40° C., preferably T_{mII}≧50° C., and even more preferably 40° C.≦T_{mII}≦90° C.

According to an embodiment of predilection, the hydrophilic polymer I is chosen from:

Ia copolymers of (meth)acrylic acid and of (meth)acrylic acid alkyl ester, and mixtures thereof;

Ib cellulose derivatives, preferably cellulose acetates, cellulose phthalates, cellulose succinates, and mixtures thereof, and even more preferably hydroxypropylmethylcellulose phthalates, hydroxypropyl-methylcellulose acetates, hydroxypropylmethylcellulose succinates, and mixtures thereof;

and mixtures thereof.

The polymers I which are even more preferred are copolymers of (meth)acrylic acid and of (meth)acrylic acid alkyl (e.g. C₁-C₆ alkyl) esters. These copolymers are, for example, of the type such as those sold by the company Rohm Pharma Polymers under the registered trademarks EUDRAGIT®, of the L and S series (such as, for example, EUDRAGIT® L100, S100, L30 D-55 and L100-55). These copolymers are anionic enteric copolymers which are soluble in an aqueous medium at pHs greater than those encountered in the stomach.

Still according to the embodiment of predilection, the compound II is chosen from the group of following products:

II.a plant waxes taken by themselves or as mixtures with one another;

II.b hydrogenated plant oils taken by themselves or as a mixture with one another;

II.c mono- and/or di- and/or triesters of glycerol and of at least one fatty acid;

II.d mixtures of monoesters, of diesters and of triesters of glycerol and of at least one fatty acid;

II.e and mixtures thereof.

Even more preferably, the compound II is chosen from the group of the following products: hydrogenated cottonseed oil, hydrogenated soybean oil, hydrogenated palm oil, glyceryl behenate, hydrogenated castor oil, tristearin, tripalmitin, trimyristin, yellow wax, hard fat or fat which can be used as bases for suppositories, anhydrous dairy fats, lanolin, glyceryl palmitostearate, glyceryl stearate, lauryl macrogolglycerides, cetyl alcohol, polyglyceryl diisostearate, diethylene glycol monostearate, ethylene glycol monostearate, omega 3, and any mixture thereof,

preferably from the subgroup of the following products: hydrogenated cottonseed oil, hydrogenated soybean oil, hydrogenated palm oil, glyceryl behenate, hydrogenated castor oil, tristearin, tripalmitin, trimyristin, and any mixture thereof.

In practice, and without this being limiting, the compound II is preferably chosen:

from the group of products sold under the following trademarks: Dynasan®, Cutina®, Hydrobase®, Dub®, Castorwax®, Croduret®, Compritol®, Sterotex®, Lubritab®, Apifil®, Akofine®, Softtisan®, Hydrocote®, Livopol®, Super Hartolan®, MGLA®, Corona®, Protalan®, Akosoft®, Akosol®, Cremao®, Massupol®, Novata®, Suppocire®, Wecobee®, Witepsol®, Lanolin®, Incromega®, Estaram®, Suppoweiss®, Gelucire®, Precirol®, Emulcire®, Plurol Diisostearique®, Geleol®, Hydrine®, Monthyle®, and mixtures thereof;

and also from the group of additives for which the codes are the following: E 901, E 907, E 903, and mixtures thereof;

and, preferably, from the group of products sold under the following trademarks: Dynasan® P60, Dynasan® 114, Dynasan® 116, Dynasan® 118, Cutina® HR, Hydrobase® 66-68, Dub® HPH, Compritol® 888, Sterotex® NF, Sterotex® K, Lubritab®, and mixtures thereof.

According to another advantageous characteristic of the invention, the coating which allows the modified release of the ARB is free of talc.

Advantageously, the coating of the microparticles can comprise, in addition to the essential constituents I and II, other conventional ingredients known to those skilled in the art, such as, in particular:

dyes,

plasticizers, for instance dibutyl sebacate,

hydrophilic compounds, for instance cellulose and its derivatives or polyvinylpyrrolidone and its derivatives,

and mixtures thereof.

Without this being limiting, and according to an even more preferred embodiment, the coating of the microparticles with modified release of ARB comprises a single composite I-II film coating.

Other details and examples of compositions and of methods for obtaining the microparticles according to the second embodiment of the invention are given in WO-A-03/030878, the content of which is integrated into the present disclosure by way of reference.

In quantitative terms, the coating monolayer can represent, for example, at most 40%, preferably at most 30% by weight of the microparticles. Such a limited amount of coating makes it possible to produce galenic units which each contain a high dose of active ingredient, without exceeding a size that would be unacceptable with regard to swallowing. This can only improve the compliance with and therefore the success of the treatment.

According to a third embodiment of the invention, the oral pharmaceutical form according to the invention comprises at least two populations of microparticles with modified release of ARB. Each population of microparticles with modified release of ARB can be in accordance with the first or with the second embodiment of the invention.

According to a variant -2i- of the second embodiment of the invention combined with the third embodiment, the oral pharmaceutical form according to the invention comprises at least two populations of microparticles with different dissolution profiles, for at least one pH value of between 1.4 and 7.4.

According to a variant -2ii- of the second embodiment of the invention combined with the third embodiment, the oral pharmaceutical form according to the invention comprises at least two populations of microparticles with modified release of ARB which differ by virtue of their respective triggering pHs.

According to yet another variant -2iii- of the second embodiment of the invention combined with the third embodiment, the oral pharmaceutical form according to the invention comprises at least two populations of microparticles with modified release of ARB which differ by virtue of their respective triggering times.

According to a fourth embodiment of the invention, the oral pharmaceutical form according to the invention comprises at least one population of microparticles with modified release of ARB and at least one population of microgranules with immediate release of ARB.

According to a variant -2iv- of the second embodiment of the invention combined with the fourth embodiment, the oral pharmaceutical form according to the invention comprises:

at least one population of microgranules with immediate release of ARB;

at least one population P1 of microparticles with modified release of ARB, and

at least one population P2 of microparticles with modified release of ARB;

and, moreover, the respective triggering pHs of P1 and of P2 differ by at least 0.5 pH unit, preferably by at least 0.8 pH unit, and even more preferably by at least 0.9 pH unit.

Advantageously, the respective triggering pHs of the various populations of microparticles with modified release of ARB are between 5 and 7.

According to a variant -2v- of the second embodiment of the invention combined with the fourth embodiment, the oral pharmaceutical form according to the invention comprises:

at least one population of microgranules with immediate release of ARB;

at least one population P1′ of microparticles with modified release of ARB, the triggering pH of which is equal to 5.5; and

at least one population P2′ of microparticles with modified release of ARB, the triggering pH of which is between 6.0 inclusive and 6.5 inclusive.

The populations P1, P2, P1′ and P2′ of the variants -2iv- and -2v- of the 2nd embodiment comprise microparticles with modified release of ARB, obtained in accordance with the 2nd embodiment of the invention.

To illustrate the variants according to which microunits with immediate release of ARB are present in the pharmaceutical form according to the invention, it can be specified that these variants can correspond to cases where this pharmaceutical form comprises, for example, at least one population of microgranules with immediate release of ARB, the behavior of which in an in vitro dissolution test is such that at least 80% of the ARB is released in 1 hour at any pH of between 1.4 and 7.4.

The oral pharmaceutical forms according to the invention can comprise at least one other active ingredient which is different than an ARB. The abbreviation AI will hereinafter denote, without distinction, one or more active ingredients which are different than an ARB.

The in vivo or in vitro release of the AI can be immediate or modified. AI can be contained in microunits of the type microgranules with immediate release of AI or in microparticles with modified release of AI.

This AI can be chosen, inter alia, from the group comprising diuretics, beta-blockers, angiotensinogen-converting enzyme inhibitors, sodium channel blockers, alpha-blockers, alpha,beta-blockers, vasodilators, alpha-antagonists and adrenergic neuronal blockers.

For further details on these additional AIs, reference may be made, for example, to the passage on page 4, line 19- page 4, line 31 of WO-A-03/035039.

The ARB can exist in several crystalline forms. The method used to prepare the pharmaceutical forms according to the invention makes it possible to conserve the initial polymorphism of the ARB.

The microparticles according to the invention can have several structures.

Thus, according to a first structural form of the microparticles, at least some of the microparticles with modified release of ARB of the oral pharmaceutical form each comprise:

a nucleus containing ARB, and

at least one coating which coats the nucleus and allows the modified release of the ARB.

According to a second structural form of the microparticles, at least some of said microparticles with modified release of ARB of the oral pharmaceutical form each comprise:

a nucleus comprising:
a neutral core, and
at least one active layer comprising the ARB and coating the neutral core,

and at least one coating which coats the nucleus and allows the modified release of the ARB.

Advantageously, the proportion of ARB in the microunits (expressed as % by weight on a dry basis relative to the total mass of the microunits) is between 5 and 80, preferably between 10 and 70, and even more preferably between 15 and 60.

According to one possibility, the microgranules with immediate release of ARB are uncoated nuclei of microparticles with modified release of ARB.

As regards the preparation of the microparticles according to the invention, this goes back to microencapsulation techniques accessible to those skilled in the art, the principles of which are summarized in the article by C. DUVERNEY and J. P. BENOIT in “L'actualite chimique” [Chemistry News], December 1986. More specifically, the technique under consideration is microencapsulation by film-coating, which produces individualized “reservoir” systems as opposed to matricial systems.

For further details, reference will be made to patent EP-B-0 953 359.

The particles of ARB of desired particle size necessary for producing the microparticles according to the invention can be crystals of ARB which is pure and/or which has undergone a pretreatment by one of the conventional techniques in the field, for instance granulation, in the presence of at least one conventional binder and/or of an agent which modifies the intrinsic solubility characteristics of the ARB. The ARB can, for example, be deposited onto the nucleus by means of the techniques known to those skilled in the art, for example the fluidized air bed “spray coating” technique, or formulated by wet granulation, compacting, extrusion-spheronization, etc.

Advantageously, the oral pharmaceutical form according to the invention is in the form of a once-daily oral dose comprising from 1000 to 500 000 microunits containing ARB.

More specifically, the oral pharmaceutical form according to the invention can be in the form of a once-daily oral dose comprising from 1000 to 500 000 microparticles with modified release of ARB.

The oral pharmaceutical form according to the invention can be provided in particular in the form of a sachet of microparticle powder, of a liquid suspension of microparticles, of a tablet obtained from microparticles, or of a gel capsule containing microparticles.

According to another of its subjects, the invention relates to the use of the microparticles with modified release of ARB as defined above and, optionally, of the microgranules with immediate release of ARB as defined above, for the preparation of pharmaceutical or dietetic, microparticulate oral galenic forms, preferably in the form of tablets, advantageously orodispersible tablets, of powders or of gel capsules.

According to yet another of its subjects, the invention relates to the microparticles per se as defined above.

According to yet another of its subjects, the invention relates to a method for the therapeutic treatment of hypertension, characterized in that it consists in administering, preferably as a once-daily oral dose, the pharmaceutical form as defined above.

### DESCRIPTION OF THE FIGURES

FIG. 1 represents the in vitro dissolution profile at pH 6.8 of the microparticles with modified release of eprosartan according to example 2.

FIG. 2 represents the in vitro dissolution profiles at pH 1.4 and 7.1 of the microparticles with modified release of candesartan cilexetil according to example 4.

FIG. 3 represents the in vitro dissolution profiles at pH 1.4 and 7.1 of the tablets with modified release of candesartan cilexetil according to example 5.

In all the figures, the dissolution profile corresponds to the percentage by weight of losartan dissolved (D) as a function of time (T) in hours.

The dissolution profiles are produced according to the indications of the European Pharmacopeia, 4th edition, entitled: “Dissolution test for solid oral forms”: type II dissolutest carried out under SINK conditions, maintained at 37° C. and stirred at 100 rpm.

### EXAMPLES

In the following examples, the excipients are denoted by their trade name. The correspondence between the trade name and the chemical name will be found in the following table.

| Trade name | Chemical name/monograph |
|---|---|
| Cremophr RH 40 | Macrogolglycerolhydroxystearate |
| Klucel EF | Hydroxypropylcellulose |
| Plasdone K29/32 | Povidone |
| Lutrol F-68 | Poloxamer |
| Kollidon CL-M | Crospovidone |
| Eudragit L100-55 | Poly(methacrylic acid, ethyl acrylate) 1:1 |
| Eudragit S100 | Poly(methacrylic acid, methyl methacrylate) 1:2 |

### Example 1

### Preparation of Granules of Eprosartan

810 g of eprosartan, 10 g of Cremophor RH 40® (BASF) and 80 g of Klucel EF® (Aqualon) are dispersed in 3000 g of purified water. The suspension is sprayed onto 100 g of neutral microspheres (Asahi-Kasei) in a Glatt GPCG1 spray coater.

The granulated material obtained has an eprosartan concentration of 81%.

### Example 2

### Preparation of Microparticles of Eprosartan

62 g of ethylcellulose (Ethocel 20 Premium®/Dow), 18 g of Plasdone K29/32® (ISP), 14 g of Lutrol F-68® (BASF) and 6 g of castor oil are solubilized in a mixture composed of 60% of isopropanol and 40% of acetone. This solution is sprayed onto 900 g of granules of eprosartan (prepared in example 1).

The microparticles obtained are then placed in a gelatin gel capsule of size 01. The dose of eprosartan per gel capsule was fixed, in this test, at 400 mg (i.e. 550 mg of microparticles). This gel capsule constitutes the final form of the medicament.

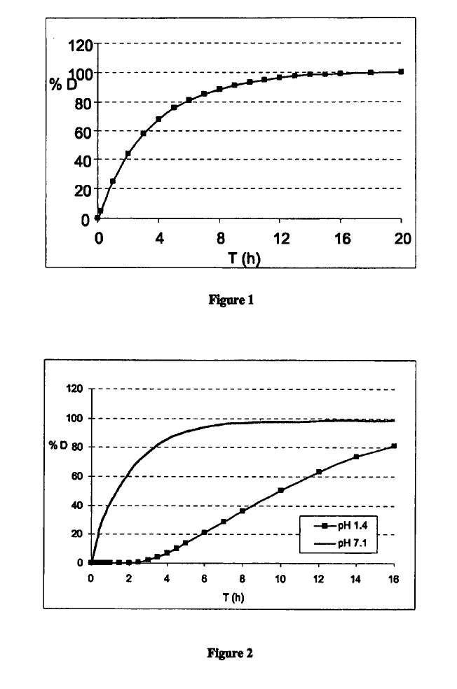

The gel capsule containing the microparticles was tested in a type II dissolutest in accordance with the Pharmacopeia at 37° C. and with stirring at 100 rpm, at pH 6.8 (0.05M KH₂PO₄/NaOH). See FIG. 1.

It is noted that the release of the eprosartan is sustained over a period of approximately 6 hours, which makes it possible, during the administration of such a medicament, to increase the bioabsorption times.

### Example 3

### Preparation of Granules of Candesartan Cilexetil

500 g of candesartan cilexetil, 80 g of Klucel EF® (Aqualon), 40 g of Lutrol F-68° and 180 g of Kollidon CL-M® (BASF) are dispersed in 3000 g of purified water. The suspension is sprayed onto 200 g of neutral microspheres (Asahi-Kasei) in a Glatt GPCG1 spray coater.

The granulation material obtained has a candesartan cilexetil concentration of 50%.

### Example 4

### Preparation of Microparticles of Candesartan Cilexetil

100 g of hydrogenated cottonseed oil (Penwest), 50 g of Eudragit® L100-55 (Rohm) and 100 g of Eudragit® S100 (Rohm) are dissolved under hot conditions in ethanol.

The solution is sprayed onto 750 g of granules of candesartan cilexetil (prepared in example 3).

The microparticles obtained are then placed in a gelatin gel capsule of size 4. The dose of candesartan cilexetil per gel capsule was fixed, in this test, at 32 mg (i.e. 85 mg of microparticles). This gel capsule constitutes the final form of the medicament.

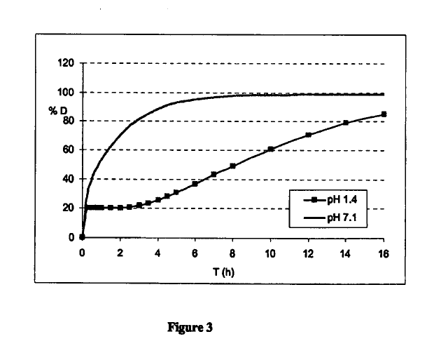

The gel capsule containing the microparticles was tested in a type II dissolutest in accordance with the Pharmacopeia at 37° C. and with stirring at 100 rpm, at pH 1.4 (HCl) and at pH 7.1. See FIG. 2.

It is noted that the release of the candesartan cilexetil is delayed and sustained, which makes it possible, during the administration of such a medicament, to increase the bioabsorption times.

### Example 5

### Tablet of Candesartan Cilexetil

12 g of granules obtained in example 3, 70 g of microparticles obtained in example 4, 70 g of PEG 6000, 100 g of dextrose, 50 g of crospovidone and 8 g of magnesium stearate are mixed using an ERWEKA laboratory mixer.

Tablets composed of 310 mg of the above mixture are prepared using a KORSCH tablet press. These tablets constitute the final form of the medicament. The disintegration time of these tablets is less than 30 minutes. After disintegration, a divided microparticulate system is again obtained.

The tablets thus prepared were tested in a type II dissolutest in accordance with the Pharmacopeia at 37° C. and with stirring at 100 rpm, at pH 1.4 (HCl) and at pH 7.1. See FIG. 3.

It is noted that the release of the candesartan cilexetil is delayed and sustained. The fact of being able to readily combine, in the same dosage, an immediate-release fraction and a delayed and sustained-release fraction makes it possible to optimize the plasma profile.

What is claimed is:

1. An oral pharmaceutical form with modified release of ARB, characterized
in that it comprises a plurality of microunits containing ARB,
in that the average diameter (Dm in μm) of the microunits is between 50 and 1000, preferably 100 and 600, and even more preferably between 150 and 500,
and in that it makes it possible to obtain, after one intake, a plasma profile defined as follows:

| | |
|---|---|
| | C18 h* ≦ C18 h |
| preferably | 1.5 × C18 h* ≦ C18 h ≦ Cmax*/1.5 |
| and even more | 2.0 × C18 h* ≦ C18 h ≦ Cmax*/1.5 |
| preferably | |

with
C18 h representing the plasma concentration of ARB, 18 h after the intake,
C18 h* representing the plasma concentration of ARB obtained under the same conditions as C18 h, with a reference immediate-release oral pharmaceutical form, containing the same dose of ARB,
Cmax representing the maximum plasma concentration of ARB after the intake,
Cmax* representing the maximum plasma concentration of ARB obtained under the same conditions as Cmax, with a reference immediate-release oral pharmaceutical form, containing the same dose of ARB.

2. The pharmaceutical form as claimed in claim 1, characterized in that it makes it possible to obtain, after one intake, a plasma profile defined as follows:

| | |
|---|---|
| | C18 h* ≦ C18 h |
| preferably | 1.5 × C18 h* ≦ C18 h ≦ Cmax*/1.5 |
| and even more | 2.0 × C18 h* ≦ C18 h ≦ Cmax*/1.5 |
| preferably | |
| and | 1.1.Tmax* ≦ Tmax |
| preferably | 1.2.Tmax* ≦ Tmax |
| and more preferably | 1.5.Tmax* ≦ Tmax |
| even more preferably | 1.7.Tmax* ≦ Tmax ≦ 6.Tmax |

with
C18 h representing the plasma concentration of ARB, 18 h after the intake,
C18 h* representing the plasma concentration of ARB obtained under the same conditions as C18 h, with a reference immediate-release oral pharmaceutical form, containing the same dose of ARB,
Cmax representing the maximum plasma concentration of ARB after the intake,
Tmax representing the time which has elapsed after the intake and which corresponds to Cmax,
Cmax* representing the maximum plasma concentration of ARB obtained under the same conditions as Cmax, with a reference immediate-release oral pharmaceutical form, containing the same dose of ARB,
Tmax* representing the time which has elapsed after the intake and which corresponds to Cmax*.

3. The oral pharmaceutical form as claimed in claim 1 or 2, characterized in that at least some of the microunits are microparticles individually consisting of a nucleus which comprises ARB and which is coated with at least one coating which allows the modified release of the ARB.

4. The oral pharmaceutical form as claimed in any one of the preceding claims, characterized in that at least some of the microunits that it comprises consist of microgranules with immediate release of ARB.

5. The oral pharmaceutical form as claimed in claim 4, characterized by an in vitro dissolution profile such that:
70% of the ARB is released between 1 and 24 h, preferably between 2 and 12 h, and even more preferably between 2 and 8 h, after the administration.

6. The oral pharmaceutical form as claimed in one of claims 1 or 2 and 3, characterized in that
the release of the ARB is controlled by two distinct triggering mechanisms, one being based on a variation in pH and the other allowing the release of the ARB, after a predetermined residence time in the stomach;
at a constant pH 1.4, the dissolution profile comprises a lag phase with a duration of less than or equal to 7 hours, preferably less than or equal to 5 hours, and even more preferably of between 1 and 5 hours,
and the passage from pH 1.4 to pH 7.0 results in a release phase which begins with no lag time.

7. The oral pharmaceutical form as claimed in claim 6, characterized in that its dissolution profile, measured in an in vitro dissolution test, is as indicated hereinafter:
less than 20% of the ARB is released after 2 hours at pH=1.4;
at least 50% of the ARB is released after 16 hours at pH=1.4.

8. The oral pharmaceutical form as claimed in any one of the preceding claims, characterized in that the variability CV (as %) of the area under the curve (AUC) giving the evolution of the plasma concentration of ARB, as a function of time (T) after the intake, is less than or equal to 200%, preferably to 150%, and even more preferably to 120%, of the corresponding variability CV* (as %) of the area under the curve (AUC*) giving the evolution of the plasma concentration of ARB, as a function of time (T) after the intake, under the same conditions, of a reference immediate-release oral pharmaceutical form* containing the same dose of ARB, i.e.: CV≦2.0×CV*, preferably CV≦1.5×CV*, and even more preferably CV≦1.2×CV*.

9. The oral pharmaceutical form as claimed in any one of the preceding claims, characterized in that it comprises at least two populations of microparticles as claimed in claim 3.

10. The oral pharmaceutical form as claimed in any one of the preceding claims, characterized in that it comprises at least one population of microparticles as claimed in claim 2 and at least one population of microgranules as claimed in claim 4.

11. The oral pharmaceutical form as claimed in claim 6 and, optionally, any one of claims 7 to 10, characterized in that it comprises at least two populations of microparticles with different dissolution profiles, for at least one pH value of between 1.4 and 7.4.

12. The oral pharmaceutical form as claimed in claim 6 and, optionally any one of claims 7 to 11, characterized in that it comprises at least two populations of microparticles with modified release of ARB which differ by virtue of their respective triggering pHs.

13. The oral pharmaceutical form as claimed in claim 6 and, optionally, any one of claims 7 to 12, characterized in that it comprises at least two populations of microparticles with modified release of ARB which differ by virtue of their respective triggering times.

14. The oral pharmaceutical form as claimed in claim 6 and, optionally, any one of claims 7 to 13, characterized in that it comprises:
at least one population of microgranules with immediate release of ARB;
at least one population P1 of microparticles with modified release of ARB, and
at least one population P2 of microparticles with modified release of ARB;
and in that the respective triggering pHs of P1 and P2 differ by at least 0.5 pH unit, preferably by at least 0.8 pH unit, and even more preferably by at least 0.9 pH unit.

15. The oral pharmaceutical form as claimed in claim 6 and, optionally, any one of claims 7 to 14, characterized in that the respective triggering pHs of the various populations of microparticles with modified release of ARB are between 5 and 7.

16. The oral pharmaceutical form as claimed in claim 6 and, optionally, any one of claims 7 to 15, characterized in that it comprises:
at least one population of microgranules with immediate release of ARB;
at least one population P1′ of microparticles with modified release of ARB, the triggering pH of which is equal to 5.5; and
at least one population P2′ of microparticles with modified release of ARB, the triggering pH of which is equal to 6.0 or 6.5.

17. The oral pharmaceutical form as claimed in any one of claims 4 to 16, characterized in that it comprises at least one population of microgranules with immediate release of ARB, the behavior of which in an in vitro dissolution test is such that at least 80% of the ARB is released in 1 hour at any pH of between 1.4 and 7.4.

18. The oral pharmaceutical form as claimed in one of claims 3 to 17, characterized in that at least some of the microparticles with modified release of ARB each comprise:
a nucleus containing ARB, and
at least one coating which coats the nucleus and allows the modified release of the ARB.

19. The oral pharmaceutical form as claimed in any one of claims 3 to 17, characterized in that at least some of said microparticles with modified release of ARB each comprise:
a nucleus comprising:
a neutral core,
at least one active layer comprising the ARB and coating the neutral core,
and at least one coating which coats the nucleus and allows the modified release of the ARB.

20. The oral pharmaceutical form as claimed in any one of the preceding claims, characterized in that the proportion of ARB in the microunits (expressed as % by weight on a dry basis relative to the total mass of the microunits) is between 5 and 80, preferably between 10 and 70, and even more preferably between 15 and 60.

21. The oral pharmaceutical form as claimed in claim 4 and, optionally, any one of claims 5 to 20,
characterized in that the microgranules with immediate release of ARB are uncoated nuclei of microparticles as claimed in claim 4.

22. The oral pharmaceutical form as claimed in any one of the preceding claims, characterized in that it is in the form of a once-daily oral dose comprising from 1000 to 500 000 microunits containing ARB.

23. The oral pharmaceutical form as claimed in any one of the preceding claims, characterized in that it is in the form of a once-daily oral dose comprising from 1000 to 500 000 microparticles with modified release of ARB.

24. The oral pharmaceutical form as claimed in any one of the preceding claims, characterized in that it is in the form of a sachet of microunit powder, of a liquid suspension of microparticles, of a tablet obtained from microunits, or of a gel capsule containing microunits.

25. The use of the microparticles with modified release of ARB as defined in any one of claims 3 to 24 and, optionally, of the microgranules with immediate release of ARB as defined in any one of claims 4 to 24, for the preparation of pharmaceutical or dietetic, microparticulate oral galenic forms, preferably in the form of tablets, advantageously orodispersible tablets, of powders or of gel capsules.

26. The use of the microparticles with modified release of ARB as defined in any one of claims 3 to 24 and, optionally, of the microgranules with immediate release of ARB as defined in any one of claims 4 to 24, for the preparation of a therapeutically safe, microparticulate oral pharmaceutical form designed in such a way that, once said pharmaceutical form has been ingested, the microparticles that it contains are dispersed and individualized when they reach the stomach, which allows these microparticles to be subjected to a regular and gradual gastric emptying, whether the patient is in the fed or unfed state at the time of intake, thus guaranteeing a release of ARB in its gastrointestinal window of bioabsorption.

27. The microparticle as defined in any one of the preceding claims.
